SEQUENCE LISTING (7 sequences)

SEQ ID NO: 1 - protein, 774 aa, Arabidopsis thaliala
MSCYNKALLIGNKVVVILVFLLCLVHSSESLRPLFACDPANGLTRTLRFCRANVPIHVRVQDLLGRLTLQEKIRNLVNNAAAVPRLGIGGYEWWSEALHGISDVGPGAKFGGAFPGATSFPQVITTAASFNQSLWEEIGRVVSDEARAMYNGGVAGLTYWSPNVNILRDPRWGRGQETPGEDPIVAAKYAASYVRGLQGTAAGNRLKVAACCKHYTAYDLDNWNGVDRFHFNAKVTQQDLEDTYNVPFKSCVYEGKVASVMCSYNQVNGKPTCADENLLKNTIRGQWRLNGYIVSDCDSVDVFFNQQHYTSTPEEAAARSIKAGLDLDCGPFLAIFTEGAVKKGLLTENDINLALANTLTVQMRLGMFDGNLGPYANLGPRDVCTPAHKHLALEAAHQGIVLLKNSARSLPLSPRRHRTVAVIGPNSDVTETMIGNYAGKACAYTSPLQGISRYARTLHQAGCAGVACKGNQGFGAAEAAAREADATVLVMGLDQSIEAETRDRTGLLLPGYQQDLVTRVAQASRGPVILVLMSGGPIDVTFAKNDPRVAAIIWAGYPGQAGGAAIANIIFGAANPGGKLPMTWYPQDYVAKVPMTVMAMRASGNYPGRTYRFYKGPVVFPFGFGLSYTTFTHSLAKSPLAQLSVSLSNLNSANTILNSSSHSIKVSHTNCNSFPKMPLHVEVSNTGEFDGTHTVFVFAEPPINGIKGLGVNKQLIAFEKVHVMAGAKQTVQVDVDACKHLGVVDEYGKRRIPMGEHKLHIGDLKHTILVQPQL

SEQ ID NO: 2 - DNA, 2325 bp, Arabidopsis thaliala
atgtcttgttataataaagcactattgatcggcaacaaagtcgtcgttatacttgtattcctcttatgtttggttcactcgtcagagtcacttcgaccactgtttgcatgtgatccagcaaacgggttaacccggacgctccggttctgtcgggccaatgtaccgatccatgtgagagttcaagatttgctcggaaggctcacgttgcaggagaagatccgcaacctcgtcaacaatgctgccgccgtaccacgtctcggtattggaggctatgagtggtggtccgaggctctccacggcatttccgacgttggtccaggcgctaagttcggtggtgcttttcccggtgccaccagcttccctcaggtcatcaccaccgcagcttctttcaaccagtctctatgggaagagatcggacgggtggtgtctgatgaggcaagagctatgtacaatggtggcgtggccggtctgacatattggagtccgaatgtgaatatcttgagggacccgcggtggggccgaggccaagaaactcccggagaagatcctatcgttgccgcaaaatatgccgccagctacgtccggggacttcagggtactgctgccggtaaccgccttaaagtcgccgcatgttgcaaacattacactgcttatgatcttgataattggaatggcgtcgaccgtttccacttcaacgctaaggtcacccaacaagatttagaggacacatacaacgtgccattcaaatcatgtgtttacgaaggaaaagtagcgagtgtaatgtgttcgtacaaccaagtcaatggaaagcccacatgtgctgatgaaaatctcttaaagaacactattcgtggtcaatggcgtctcaatgggtacattgtctcagattgtgactctgttgatgttttcttcaaccaacaacattacactagcactccggaagaagccgccgccagatccattaaagccggtttggacttggactgcgggccgtttttggcgattttcacggaaggtgcagtgaagaaaggattgttaacggagaatgacatcaatttagcacttgctaatacattaacagtccaaatgagacttggtatgtttgatggtaaccttgggccgtacgctaatcttgggccaagagatgtttgtactccggcccataaacatttagctcttgaagcagcccatcaagggattgttcttctcaaaaactctgctcgctctcttccactctcccccagacgccaccgcaccgtcgccgtgattggtccaaactccgacgtcactgagactatgatcggcaactatgcagggaaagcatgcgcctatacgtcgccgttgcaagggatttcaagatacgcgaggacacttcaccaagctggctgtgccggcgtggcttgcaaagggaaccaaggatttggtgcagcggaggcagcggcgcgtgaagccgacgcgacggttcttgtgatgggattagatcagtcgatagaggcagagacacgagatcgaaccgggcttctcttaccgggttatcaacaagacctagtgacccgtgtagctcaagcttctagaggtccagtcattttggtccttatgagtggtggaccaatcgatgtaaccttcgctaagaatgatcctcgtgttgctgccatcatttgggctgggtatccgggtcaagcgggtggagctgccatcgccaatatcatctttggtgctgctaatcccggaggaaaactaccaatgacatggtatccacaagattacgtggccaaagtgccaatgacggtaatggccatgagagcatccggtaattatccaggaaggacatacagattctacaaaggtccagtagtatttccatttgggttcggtttaagttacactaccttcactcatagtttggccaaaagcccattggcccaactatcagtttcactctccaatctcaactctgccaataccattctcaactcttcatcacactccatcaaagtgtctcacaccaactgcaattcatttccgaaaatgccccttcacgtcgaagtatcaaacacaggtgaattcgatggaacacacacggtgtttgtatttgctgagccgccgataaacggaataaaaggattgggtgtgaacaaacaattgatagcgttcgagaaggttcatgtcatggcaggggcaaaacagaccgttcaagttgatgttgatgcttgcaagcatcttggtgtagtggatgagtatggaaagaggagaatcccaatgggtgagcataagctgcacattggtgaccttaaacatactattttggtccaaccgcaactttga

SEQ ID NO: 3 - DNA, 20 bp, artificial sequence
  primer
agttgatgttgatgcttgca

SEQ ID NO: 4 - DNA, 20 bp, artificial sequence
  primer
aagttgcggttggaccaaaa

SEQ ID NO: 5 - DNA, 24 bp, artificial sequence
  linker
  misc_feature (6)..(24): n is a, c, g, or t
gattgnnnnnnnnnnnnnnnnnnn

SEQ ID NO: 6 - DNA, 24 bp, artificial sequence
  linker
  misc_feature (6)..(24): n is a, c, g, or t
tggcgnnnnnnnnnnnnnnnnnnn

SEQ ID NO: 7 - DNA, 24 bp, artificial sequence
  linker
  misc_feature (2)..(20): n is a, c, g, or t
cnnnnnnnnnnnnnnnnnnncaaa

(19) United States
(12) Patent Application Publication
(10) Pub. No.: US 2022/0177904 A1
(43) Pub. Date: Jun. 9, 2022

(54) USE OF BXL GENE OR PROTEIN ENCODED THEREBY

(71) Applicant: CAS CENTER FOR EXCELLENCE IN MOLECULAR PLANT SCIENCES, Shanghai (CN)

(72) Inventors: Huiming ZHANG, Shanghai (CN); Danxia HE, Shanghai (CN)

(73) Assignee: CAS CENTER FOR EXCELLENCE IN MOLECULAR PLANT SCIENCES, Shanghai (CN)

(21) Appl. No.: 17/427,647

(22) PCT Filed: Sep. 1, 2020
(86) PCT No.: PCT/CN2020/071199

§ 371 Date: Feb. 18, 2022

(30) Foreign Application Priority Data
Jan. 31, 2019 (CN) 201910098153.7

(51) Int. Cl. C12N 15/82; C12N 9/42

(52) U.S. Cl. CPC C12N 15/8279; C12Y 302/01037; C12N 9/2434; C12N 15/8255

(57) ABSTRACT

Disclosed is a use of the BXL gene or a protein encoded thereby. Specifically, when the expression of the BXL gene or a protein encoded thereby is inhibited, traits of plants can be significantly improved, comprising: (i) enhancing the stress resistance of plants; and/or (ii) the resistance to pathogens; and/or (iii) reducing lignin content and increasing fiber and pectin content. In addition, inhibitors of the BXL gene or a protein encoded thereby can also be used in feed compositions, and are used for improving feed palatability.

### TECHNICAL FIELD

The present invention relates to the field of agriculture, in particular to the application of a BXL gene or a coded protein thereof.

### BACKGROUND

Plants experience various biotic or abiotic stresses during their growth. Drought and pathogenic bacteria, as the two main factors that harm plant growth, have been widely focused by people.

However, the current main methods for improving drought and pathogenic bacteria are conventional irrigation techniques and spraying pesticides. Conventional irrigation techniques include: digging ditches, building reservoirs, barrages, pilotage, or now commonly used water-saving irrigation techniques in greenhouses—drip irrigation. However, these conventional methods for improving drought have many shortcomings, such as, it is difficult to implement, consuming more manpower and material resources, it is uneconomical and has poor engineering modification, and has limited scope for improving drought, and has limited application scope, etc. People use the method of spraying pesticides to realize the improvement of pathogenic bacteria. Although straightforward, there are many shortcomings, such as: pesticide residues, endangering human health, causing environmental pollution, polluting water resources, destroying air cleanliness, and contrary to the green concept advocated by mankind.

Therefore, there is an urgent need in this field to develop a new gene that can help plants resist external stress, especially drought stress and pathogenic bacteria.

### SUMMARY OF THE INVENTION

The purpose of the present invention is to provide a new gene that can help plants resist external stress, especially drought stress and pathogenic bacteria.

In a first aspect of the present invention, it provides a use of an inhibitor of a BXL gene or an encoded protein thereof for the improvement of plant traits; or preparing a composition or preparation for the improvement of plant traits.

In another preferred embodiment, the improvement of plant traits include:

(i) enhancing plant stress resistance; and/or

(ii) anti-pathogenic bacteria; and/or

(iii) reducing the lignin content, increasing the fiber and pectin content.

In another preferred embodiment, the stress resistance is selected from the group consisting of drought resistance, salt resistance, osmotic pressure resistance, heat resistance, and a combination thereof.

In another preferred embodiment, the pathogenic bacteria is selected from the group consisting of Pseudomonas syringae, Pseudomonas syring pv. maculicola, and a combination thereof.

In another preferred embodiment, the pathogenic bacteria is selected from the group consisting of: Pseudomonas syringae tomato pathogenic strain DC3000, Pseudomonas syringae tomato pathogenic strain T1, Pseudomonas syringae tomato pathogenic strain 3435, Pseudomonas syring pv. maculicola 4326. Pseudomonas syring pv. maculicola 4981, and a combination thereof.

In another preferred embodiment, the composition or preparation is also used for one or more purposes selected from the group consisting of:

(a) delaying seed germination;

(b) delaying flowering time;

(c) reducing the stomatal aperture;

(d) enhancing resilience;

(e) enhancing the ability to resist pathogenic bacteria;

(f) improving the palatability as feed.

In another preferred embodiment, the composition includes an agricultural composition.

In another preferred embodiment, the formulation includes an agricultural formulation.

In another preferred embodiment, the composition comprises (a) an inhibitor of a BXL gene or an encoded protein thereof; and (b) an agronomically acceptable carrier.

In another preferred embodiment, the dosage form of the composition or preparation is selected from the group consisting of: solution, emulsion, suspension, powder, foaming agent, paste, granules, aerosol, and a combination thereof.

In another preferred embodiment, the inhibitor is selected from the group consisting of an antisense nucleic acid, antibody, small molecule compound, Crispr reagent, and volatile matter of bacterial (including acetoin, di-, tri-butanediol, di-, tri-butanedione, isoamylol, n-butyl alcohol), siRNA, shRNA, miRNA, small molecule ligand, and a combination thereof.

In another preferred embodiment, the inhibitor is selected from the group consisting of abscisic acid (ABA), salt (NaCl), salicylic acid (SA), mannitol (Mannitol), volatile matter of Bacillus amyloliquefaciens GB03 (including acetoin, di-, tri-butanediol, di-, tri-butanedione, isoamylol, n-butyl alcohol), and a combination thereof.

In another preferred embodiment, the composition further includes other substances that improve the palatability of feed.

In another preferred embodiment, the other substances that improve the palatability of feed are selected from the group consisting of: cellulose hydrolase, pectin synthase, pectin additive, galactase, ethylene molecule, abscisic acid, and a combination thereof.

In another preferred embodiment, the composition further includes other substances that enhance plant resistance to stress.

In another preferred embodiment, the other substances that enhance plant resistance to stress are selected from the group consisting of: abscisic acid, analogs of abscisic acid, callose, proline, and a combination thereof.

In another preferred embodiment, the other anti-pathogenic substances are selected from the group consisting of alkaloids, flavonoids, tannins, phenylpropanoids, and combinations thereof.

In another preferred embodiment, the composition further includes other substances that reduce the content of lignin and increase the content of fiber and pectin.

In another preferred embodiment, the other substances that reduce the content of lignin and increase the content of fiber and pectin are selected from the group consisting of: glycosyl hydrolase, arabinosidase, xylosidase, and a combination thereof.

In another preferred embodiment, the plant includes a monocotyledonous plant and a dicotyledonous plant.

In another preferred embodiment, the plant includes a herbaceous plant and a woody plant.

In another preferred embodiment, the herbaceous plant is selected from the group consisting of Solanaceae, Gramineous plant, Leguminous plant, Cruciferous plant, and a combination thereof.

In another preferred embodiment, the woody plant is selected from the group consisting of Actinidiaceae, Rosaceae, Moraceae, Capparaceae, Rutaceae, Malvaceae, Rosaceae, Hibiscus, and a combination thereof.

In another preferred embodiment, the plant is selected from the group consisting of a cruciferous plant, gramineous plant, leguminous plant, Solanaceae, Actinidiaceae, Malvaceae, Paeoniaceae, Rosaceae, Liliaceae, and combinations thereof.

In another preferred embodiment, the plant is selected from the group consisting of Arabidopsis, alfalfa, apple, Camelina sativa, soybean, rice, oilseed rape, radish, pepper, cherry, Jujube, cabbage, Cleome hassleriana, citrus, durian, and a combination thereof.

In another preferred embodiment, the BXL gene is selected from the group consisting of: BXL1, BXL2, BXL3, BXL4, BXL7, AT5G10560, AT3G19620, AT5G09700, and a combination thereof.

In another preferred embodiment, the BXL gene includes wild-type BXL gene and mutant BXL gene.

In another preferred embodiment, the mutant includes a mutant form in which the function of the encoded protein is not changed after the mutation (that is, the function is the same or substantially the same as that of the wild-type encoded protein).

In another preferred embodiment, the polypeptide encoded by the mutant BXL gene is the same or substantially the same as the polypeptide encoded by the wild-type BXL gene.

In another preferred embodiment, the mutant BXL gene includes compared with the wild-type BXL gene, a polynucleotide with homology ≥80% (preferably ≥90%, more preferably ≥95%, more preferably, ≥98% or 99%).

In another preferred embodiment, the mutant BXL gene includes a polynucleotide with 1-60 (preferably 1-30, more preferably 1-10) nucleotides truncated or added at the 5′ end and/or 3′ end of the wild-type BXL gene.

In another preferred embodiment, the BXL gene includes a cDNA sequence, a genomic sequence, and a combination thereof.

In another preferred example, the BXL gene is derived from a plant, preferably from a dicotyledonous plant, more preferably, from one or more plants selected from the group consisting of Arabidopsis, alfalfa, apple, Camelina sativa, soybean, oilseed rape, radish, pepper, cherry, Jujube, cabbage, Cleome hassleriana, citrus, durian.

In another preferred embodiment, the BXL gene is selected from the group consisting of the BXL1 gene of Arabidopsis thaliana (gene accession number: AT5G49360), the xylosidase 1 gene of Camelina sativa (Camelina sativa (L.) Crantz's β-D-xylosidase 1, gene accession number: LOC104723790), xylosidase gene of alfalfa (β-D-xylosidase of Medicago Sativa Linn, gene accession number: Medtr2G034720.1), xylosidase gene of Brassica napus (β-D-xylosidase of Brassica napus L., gene accession number: LOC106365857), Rosa multiflora xylosidase 2 gene (β-D-xylosidase 2 gene of Rosa sp., gene accession number: LOC112179881), and a combination thereof.

In another preferred embodiment, the amino acid sequence of the encoded protein of the BXL gene is selected from the group consisting of:

(i) a polypeptide having the amino acid sequence as shown in SEQ ID NO.:1;

(ii) a polypeptide derived from (i), having the function of regulating agronomic traits formed by the substitution, deletion or addition of one or several (such as 1-10) amino acid residues of the amino acid sequence as shown in SEQ ID NO.:1; or (iii) a polypeptide whose amino acid sequence is greater than or equal to 90% (preferably greater than or equal to 95%, more preferably greater than or equal to 98%) homologous to the amino acid sequence as shown in SEQ ID NO.:1 and having the function of improving plant agronomic traits.

In another preferred embodiment, the nucleotide sequence of the BXL gene is selected from the group consisting of:

(a) a polynucleotide encoding the polypeptide as shown in SEQ ID NO.:1;

(b) a polynucleotide whose sequence is shown in SEQ ID NO.: 2;

(c) a polynucleotide whose nucleotide sequence has a homology of 95% (preferably ≥98%, more preferably ≥99%) with the sequence as shown in SEQ ID NO.: 2;

(d) a polynucleotide of 1-60 (preferably 1-30, more preferably 1-10) nucleotides is truncated or added at the 5′ end and/or 3′ end of the polynucleotide as shown in SEQ ID NO.: 2;

(e) A polynucleotide complementary to any of the polynucleotides as described in (a) to (d).

In a second aspect of the present invention, it provides a composition comprising:

(a) an inhibitor of a BXL gene or an encoded protein thereof; and

(b) an agronomically acceptable carrier.

In another preferred embodiment, the composition includes an agricultural composition.

In another preferred embodiment, the agricultural composition is selected from the group consisting of a feed composition, an organic fertilizer composition, a pesticide composition, and a combination thereof.

In another preferred embodiment, the feed composition includes a solid feed composition or a liquid feed composition.

In another preferred embodiment, the feed composition is a plant breeding additive.

In another preferred embodiment, the dosage form of the composition is selected from the group consisting of: solution, emulsion, suspension, powder, foaming agent, paste, granules, aerosol, and a combination thereof.

In another preferred embodiment, the composition contains 0.0001-99 wt %, preferably 0.1-90 wt % of component (a), based on the total weight of the composition.

In another preferred embodiment, the content (wt %) of the inhibitor of a BXL gene or an encoded protein thereof in the composition is 0.05%-10%, preferably, 0.1%-8%, more preferably, 0.5%-6%.

In another preferred embodiment, the inhibitor of the BXL gene or the encoded protein thereof is selected from the group consisting of a small molecule compound, antibody, volatile matter of bacterial (including acetoin, di-, tri-butanediol, di-, tri-butanedione, isoamylol, n-butyl alcohol), antisense nucleic acid, Crispr reagent, siRNA, shRNA, miRNA, small molecule ligand and a combination thereof.

In another preferred embodiment, the inhibitor of the BXL gene or the encoded protein thereof is selected from the group consisting of a small molecule compound, antibody, volatile matter of bacterial, and a combination thereof.

In another preferred embodiment, the inhibitor of the BXL gene or the encoded protein thereof is selected from the group consisting of: abscisic acid (ABA), salt (NaCl), salicylic acid (SA), Mannitol, volatile matter of Bacillus amyloliquefaciens GB03 (including acetoin, di-, tri-butanediol, di-, tri-butanedione, isoamylol, n-butyl alcohol), and a combination thereof.

In another preferred embodiment, the composition further includes other substances that improve the palatability of feed.

In another preferred embodiment, the other substances that improve the palatability of feed are selected from the group consisting of: cellulose hydrolysis, pectin synthase, pectin additive, galactase, ethylene molecule, abscisic acid, and a combination thereof.

In another preferred embodiment, the composition further includes other substances that enhance plant resistance to stress.

In another preferred embodiment, the other substances that enhance plant resistance to stress are selected from the group consisting of: abscisic acid, analogs of abscisic acid, callose, proline, and a combination thereof.

In another preferred embodiment, the other anti-pathogenic bacteria substances are selected from the group consisting of alkaloids, flavonoids, tannins, phenylpropanoids, and combinations thereof.

In another preferred embodiment, the composition also includes other substances that reduce the content of lignin and increase the content of fiber and pectin.

In another preferred embodiment, the other substances that reduce the content of lignin and increase the content of fiber and pectin are selected from the group consisting of: glycosyl hydrolase, arabinosidase, xylosidase, and a combination thereof.

In a third aspect of the present invention, it provides a use of the composition according to the second aspect of the present invention for improving a agronomic trait of a plant.

In a fourth aspect of the present invention, it provides a method for improving the palatability of feed, comprising the steps:

reducing the expression level and/or activity of the BXL gene or the encoded protein thereof in the plant, thereby improving the palatability of feed.

In another preferred embodiment, the method includes administering an inhibitor of the BXL gene or the encoded protein thereof to the plant.

In another preferred embodiment, the method includes the steps:

(i) providing a plant or plant cell; and

(ii) introducing the inhibitor of the BXL gene or the encoded protein thereof into the plant or plant cell, thereby obtaining a modified plant or plant cell.

In another preferred embodiment, the inhibitor of the BXL gene or the encoded protein thereof is selected from the group consisting of: a small molecule compound, antibody, volatile matter of bacterial (including acetoin, di-, tri-butanediol, di-, tri-butanedione, isoamylol, n-butyl alcohol), antisense nucleic acid, Crispr reagent, siRNA, shRNA, miRNA, small molecule ligand, and a combination thereof.

In another preferred embodiment, the inhibitor of the BXL gene or the encoded protein thereof is selected from the group consisting of: a small molecule compound, antibody, volatile matter of bacterial (including acetoin, di-, tri-butanediol, di-, tri-butanedione, isoamylol, n-butyl alcohol) and a combination thereof.

In another preferred embodiment, the inhibitor is selected from the group consisting of abscisic acid (ABA), salt (NaCl), salicylic acid (SA), mannitol (Mannitol), volatile matter of Bacillus amyloliquefaciens GB03 (including acetoin, di-, tri-butanediol, di-, tri-butanedione, isoamylol, n-butyl alcohol), and a combination thereof.

In another preferred embodiment, the “reducing” means that the reduction in the expression or activity of the BXL gene or the encoded protein thereof satisfies the following conditions:

the ratio of A1/A0 is ≤80%, preferably ≤50%, more preferably ≤20%, and most preferably 0-10%; wherein, A1 is the expression or activity of BXL gene or the encoded protein thereof; A0 is the expression or activity of the same BXL gene or the encoded protein thereof in a wild-type plant of the same type.

In another preferred embodiment, the “reducing” means that the expression level E1 of the BXL gene or the encoded protein thereof in the plant is 0-80%, preferably 0-60%, more preferably 0-40% of the wild type, compared with the expression level E0 of the wild-type BXL gene or the encoded protein thereof.

In another preferred embodiment, the reducing the expression or activity of the BXL gene or the encoded protein thereof in the plant is achieved by a method selected from the group consisting of gene mutation, gene knockout, gene disruption, RNA interference, Crispr technology, ZFN (Zinc Finger Endonuclease Technology), TALEN (Transcription Activator-like Effector Nuclease), and a combination thereof.

In a fifth aspect of the present invention, it provides a method for improving plant traits, comprising the steps:

reducing the expression level and/or activity of the BXL gene or the encoded protein thereof in the plant, thereby improving the plant traits.

In another preferred embodiment, the improving plant traits include:

(i) enhancing the plant resistance to stress; and/or

(ii) anti-pathogenic bacteria; and/or

(iii) reducing lignin content, increasing fiber and pectin content.

In another preferred embodiment, the improving plant traits further include:

(a) delay seed germination;

(b) delay flowering time;

(c) reduce stomatal aperture;

(d) enhance resilience;

(e) enhance the ability to resist pathogenic bacteria;

(f) improve the palatability as feed.

In another preferred embodiment, the reducing lignin content means that compared with a wild-type plant, the lignin content is reduced by ≥50%, preferably, ≥70%, more preferably, ≥80%, more preferably, ≥90%.

In another preferred embodiment, the increasing fiber and pectin content means that compared with a wild-type plant, the fiber and pectin content is increased by ≥30%, preferably, ≥40%, more preferably, ≥50%, more preferably, ≥60%.

In another preferred embodiment, the reduction of stomatal aperture means that compared with a wild-type plant, the stomatal aperture is reduced by 50%, preferably, 50%, more preferably, 60%, more preferably, 70%.

In another preferred embodiment, when the ratio of the BXL activity E1 in the plant to the wild-type BXL background activity E0 in the plant is ≤½, preferably ≤⅕, more preferably ≤ 1/10, the improved traits of the plant include:
(i) enhance the plant resistance to stress; and/or
(ii) anti-pathogenic bacteria; and/or
(iii) reduce lignin content, increasing fiber and pectin content; and/or
(iv) delay seed germination; and/or
(v) delay flowering time; and/or
(vi) reduce stomatal aperture.

In another preferred embodiment, the “reducing” means that the reduction in the expression or activity of the BXL gene or the encoded protein thereof satisfies the following conditions:

the ratio of A1/A0 is ≤80%, preferably ≤50%, more preferably ≤20%, and most preferably 0-10%; wherein, A1 is the expression or activity of BXL gene or the encoded protein thereof; A0 is the expression or activity of the same BXL gene or the encoded protein thereof in a wild-type plant of the same type.

In another preferred embodiment, the “reducing” means that the expression level E1 of the BXL gene or the encoded protein thereof in the plant is 0-80%, preferably 0-60%, more preferably 0-40% of the wild type, compared with the expression level E0 of the wild-type BXL gene or the encoded protein thereof.

In another preferred embodiment, the reducing the expression or activity of the BXL gene or the encoded protein thereof in the plant is achieved by a method selected from the group consisting of gene mutation, gene knockout, gene disruption, RNA interference, Crispr technology, ZFN (Zinc Finger Endonuclease Technology), TALEN (Transcription Activator-like Effector Nuclease), and a combination thereof.

In another preferred embodiment, the method includes administering an inhibitor of the BXL gene or the encoded protein thereof to the plant.

In another preferred embodiment, the method includes the steps:

(i) providing a plant or plant cell; and

(ii) introducing the inhibitor of the BXL gene or the encoded protein thereof into the plant or plant cell, thereby obtaining a modified (e.g., genetically modified) plant or plant cell.

In another preferred embodiment, the inhibitor of the BXL gene or the encoded protein thereof is selected from the group consisting of: a small molecule compound, antibody, volatile matter of bacterial (including acetoin, di-, tri-butanediol, di-, tri-butanedione, isoamylol, n-butyl alcohol), antisense nucleic acid, Crispr reagent, siRNA, shRNA, miRNA, small molecule ligand, and a combination thereof.

In another preferred embodiment, the inhibitor of the BXL gene or the encoded protein thereof is selected from the group consisting of: a small molecule compound, antibody, volatile matter of bacterial (including acetoin, di-, tri-butanediol, di-, tri-butanedione, isoamylol, n-butyl alcohol) and a combination thereof.

In another preferred embodiment, the inhibitor is selected from the group consisting of abscisic acid (ABA), salt (NaCl), salicylic acid (SA), mannitol (Mannitol), volatile matter of Bacillus amyloliquefaciens GB03 (including acetoin, di-, tri-butanediol, di-, tri-butanedione, isoamylol, n-butyl alcohol), and a combination thereof.

In a sixth aspect of the present invention, it provides a method for preparing a genetically engineered plant tissue or plant cell, comprising the steps:

reducing the expression and/or activity of the BXL gene or the encoded protein thereof in a plant tissue or plant cell, thereby obtaining a genetically engineered plant tissue or plant cell.

In another preferred embodiment, the method further includes introducing the inhibitor of the BXL gene or the encoded protein thereof into the plant tissue or plant cell.

In another preferred embodiment, the inhibitor of the BXL gene or the encoded protein thereof is selected from the group consisting of: a small molecule compound, antibody, volatile matter of bacterial (including acetoin, di-, tri-butanediol, di-, tri-butanedione, isoamylol, n-butyl alcohol), antisense nucleic acid, Crispr reagent, siRNA, shRNA, miRNA, small molecule ligand, and a combination thereof.

In another preferred embodiment, the inhibitor of the BXL gene or the encoded protein thereof is selected from the group consisting of: a small molecule compound, antibody, volatile matter of bacterial (including acetoin, di-, tri-butanediol, di-, tri-butanedione, isoamylol, n-butyl alcohol) and a combination thereof.

In another preferred embodiment, the inhibitor is selected from the group consisting of abscisic acid (ABA), salt (NaCl), salicylic acid (SA), mannitol (Mannitol), volatile matter of Bacillus amyloliquefaciens GB03 (including acetoin, di-, tri-butanediol, di-, tri-butanedione, isoamylol, n-butyl alcohol), and a combination thereof.

In a seventh aspect of the present invention, it provides a method for preparing a genetically engineered plant, comprising the steps:

regenerating the genetically engineered plant tissue or plant cell prepared by the method of the sixth aspect of the present invention into a plant, thereby obtaining a genetically engineered plant.

In another preferred embodiment, the method includes using RNA interference, Crispr technology, ZFN (zinc finger endonuclease technology), TALEN (transcription activator-like effector nuclease) to reduce the expression and/or activity of the BXL gene or the encoded protein thereof in a plant tissue or plant cell.

In an eighth aspect of the present invention, it provides a genetically engineered plant, which is prepared by the method according to the seventh aspect of the present invention.

It should be understood that, within the scope of the present invention, each technical feature of the present invention described above and in the following (as examples) may be combined with each other to form a new or preferred technical solution, which is not listed here due to space limitations.

### DESCRIPTION OF FIGURE

FIG. 1 shows the tissue location of the BXL1 gene in Arabidopsis. The figure clearly shows that BXL1 can be expressed in vascular tissues. In leaves, the present invention has discovered for the first time that BXL1 can be specifically expressed in stoma.

FIG. 2 shows the gene expression of BXL1 in wild-type, mutant and overexpressing plants. Quantitative PCR detects the expression level of the BXL1 gene at the transcript level of the BXL1 mutant or overexpression lines. The results show that the expression of the BXL1 gene is significantly increased in the over-expressed plants compared to the wild-type plants. However, the expression of the mutant BXL1 gene is significantly suppressed compared to the wild-type plant.

FIG. 3(A)(B), the results of the two graphs show the basic phenotype of BXL1 gene resistance to pathogenic bacteria and drought. (A) The figure shows that the BXL1 mutant plants have better resistance to pathogenic bacteria, and the degree of infection is reduced compared to the wild type. (B) The figure shows that the mutant plants can better resist the drought environment. Under the same drought conditions, compared to the wild-type plants, the mutants can maintain a higher survival rate.

FIG. 4(A)(B), the results of the two graphs show the effect of the BXL1 gene on the normal growth of plants. The figure (A) shows that the BXL1 mutant plants can have a slow seed germination rate, and the figure (B) shows that the mutant plants show a slow growth rate. In the same growth period, compared with the wild type, the BXL1 mutant shows a smaller plant morphology, and the flowering time is slower. At the beginning of fruiting, the length of the fruit pod is shorter than that of the wild type.

FIG. 5(A)(B), the results of the two graphs show that the BXL1 gene has no effect on the final biomass of a single plant and the thousand seeds weight of a single plant seed. Figure (A) shows that when the BXL1 mutant plant reaches the flowering stage, compared with the wild type, the dry weight of a single plant at the time of flowering does not change significantly. Figure (B) shows compared with the seeds harvested from a wild-type single plant, the final harvested seeds of the mutant plants show no difference in dry weight in the thousand-seed weight of the seeds.

FIG. 6 shows that the BXL1 gene regulates the opening degree of plant stomata. Compared with the wild type, the stomatal aperture in the BXL1 mutant is significantly smaller.

FIG. 7 shows that the BXL1 gene is regulated by a variety of stress factors. salicylic acid, salt, abscisic acid, mannitol, these stress factors can significantly down-regulate the expression of BXL1 gene in a specific time. The mock in the figure represents water treatment, which is used as a control group.

### DETAILED DESCRIPTION

After extensive and in-depth research, the present inventors have discovered a BXL gene or an encoded protein thereof for the first time by studying and screening a large number of plant trait sites. The protein encoded by it is a glycosyl hydrolase. When inhibiting the expression of BXL gene or an encoded protein thereof, it can significantly improve plant traits, including (i) enhancing plant resistance; and/or (ii) anti-pathogenic bacteria; and/or (iii) reducing lignin content, increasing fiber and pectin content. In addition, the present inventors have also found that inhibiting the expression of the BXL gene or an encoded protein thereof also (a) delays seed germination; (b) delays flowering time; (c) reduces stomatal aperture and the like. The present inventors have completed the present invention on this basis.

BXL Gene

The gene encoding glycosyl hydrolase is named β-D-xylosidase 1 (BXL1), which has the dual functions of encoding β-D-xylosidase and {alpha}-L-arabinfuranosidease. BXL1 exists in areas outside the cytoplasm and is specifically expressed in tissues with secondary cell wall thickening, such as vascular tissues and secondary cell walls. BXL1 belongs to one of the three members of the glycosyl hydrolase family, which controls the germination of seeds and hydrolyzes araban and xylan. When a normal seed germinates, a complete viscous fluid will be released around the seed to help soften the seed coat and promote the formation of seedlings.

BXL1 plays different roles in different tissues. It mainly plays the role of xylosidase in the stem tissues, and mainly plays the role of arabinfuranosidease during the germination of seeds.

In Arabidopsis, BXL1 has 7 homologous genes, namely BXL2, BXL3, BXL4, BXL7, AT5G10560, AT3G19620, AT5G09700, respectively.

The homology degree analysis of the similarity of protein domains shows that the homology degree of the protein encoding 7 homologous genes (BXL2, BXL3, BXL4, BXL7, AT5G10560, AT3G19620, AT5G09700) and BXL1 protein is 99%, 98%, 98%, 97%, 97%, 96%, 97%, respectively.

The study of BXL1 expression tissues has found that the BXL1 gene exists in stems, fruit pods, and vascular tissues.

The present invention conducts a more in-depth study on BXL1, and through tissue staining, it is found that the BXL1 gene can be specifically expressed in the guard cells of the stomata. Physiological and biochemical research results indicate that the BXL1 gene plays an important role in helping plants resist external stress, especially drought stress and pathogenic bacteria. This discovery not only opens a new idea for studying the genes related to the secondary cell wall in plant drought and disease resistance, but also adds new content to the function of the BXL1 gene.

As used herein, the terms “BXL gene of the present invention” and “BXL gene” can be used interchangeably, and both refer to the BXL gene or its variants derived from monocotyledonous or dicotyledonous plants (such as Arabidopsis, alfalfa, apple, etc.). In a preferred embodiment, the nucleotide sequence of the BXL gene of the present invention is shown in SEQ ID NO.:2.

Representative BXL (such as BXL1) homologous genes of other species include (but are not limited to): Arabidopsis BXL gene (BXL1), alfalfa BXL homologous gene (Medtr2G0347201 and 80% similarity with Arabidopsis), Camelina sativa BXL homologous gene (β-D-xylosidase 1 (LOC104723790) and 100% similarity with Arabidopsis), Brassica napus BXL homologous gene (β-D-xylosidase 1 (LOC106365857) and 99% similarity with Arabidopsis).

The present invention also includes a nucleic acid having 50% or more (preferably 60% or more, 70% or more, 80% or more, more preferably 90% or more, more preferably 95% or more, most preferably 98% or more, such as 99%, or 100%) homology with the preferred gene sequence of the present invention (SEQ ID NO.: 2), which can also effectively regulate the agronomic traits of plants (such as Arabidopsis thaliana, alfalfa, etc.). “Homology” refers to the level of similarity (i.e., sequence similarity or identity) between two or more nucleic acids according to the percentage of positional identity. In this article, the gene variants can be obtained by inserting or deleting regulatory regions, performing random or site-directed mutations, and the like.

In the present invention, the nucleotide sequence in SEQ ID NO.: 2 can be substituted, deleted or added one or more to generate a derived sequence of SEQ ID NO.: 2. Due to the degeneracy of the codon, even though the homology with SEQ ID NO.: 2 is low, the amino acid sequence shown in SEQ ID NO.: 1 can be basically encoded. In addition, the meaning of “the nucleotide sequence in SEQ ID NO.: 2 has been substituted, deleted or added at least one nucleotide-derived sequence” also includes a nucleotide sequence that can hybridize to the nucleotide sequence shown in SEQ ID NO.: 2 under moderately stringent conditions, and better under highly stringent conditions. These variants include (but are not limited to): deletion, insertion and/or substitution of several (usually 1-90, preferably 1-60, more preferably 1-20, most preferably 1-10) nucleotides, and adding several (usually 60 or less, preferably 30 or less, more preferably 10 or less, most preferably 5 or less) nucleotides at the 5′ and/or 3′ end.

It should be understood that although the genes provided in the examples of the present invention are derived from Arabidopsis thaliana and alfalfa, the gene sequence of BXL that is derived from other similar plants and has certain homology (conservation) with the sequence of the present invention (preferably, the sequence is shown in SEQ ID NO.: 2 (Arabidopsis)) is also included in the scope of the present invention, as long as those skilled in the art can easily isolate the sequence from other plants based on the information provided in this application after reading this application.

The polynucleotide of the present invention may be in the form of DNA or RNA. The form of DNA includes: DNA, genomic DNA or synthetic DNA. DNA can be single-stranded or double-stranded. DNA can be a coding strand or a non-coding strand. The coding region sequence encoding the mature polypeptide may be the same as the coding region sequence as shown in SEQ ID NO.: 2 or a degenerate variant.

A polynucleotide encoding a mature polypeptide includes: a coding sequence that only encodes the mature polypeptide; the coding sequence of the mature polypeptide and various additional coding sequences; the coding sequence (and optional additional coding sequence) of the mature polypeptide and non-coding sequences.

The term “polynucleotide encoding a polypeptide” may include a polynucleotide encoding the polypeptide, or a polynucleotide that also includes additional coding and/or non-coding sequences. The present invention also relates to variants of the above-mentioned polynucleotides, which encode fragments, analogs and derivatives of polynucleotide or polypeptides having the same amino acid sequence as the present invention. The variants of this polynucleotide can be naturally occurring allelic variants or non-naturally occurring variants. These nucleotide variants include substitution variants, deletion variants and insertion variants. As known in the art, an allelic variant is an alternative form of a polynucleotide, which may be a substitution, deletion or insertion of one or more nucleotides, but does not substantially change the function of the encoded polypeptide.

The present invention also relates to polynucleotides that hybridize with the aforementioned sequences and have at least 50%, preferably at least 70%, and more preferably at least 80% identity between the two sequences. The present invention particularly relates to polynucleotides that can hybridize with the polynucleotide of the present invention under stringent conditions. In the present invention, “stringent conditions” refer to: (1) hybridization and elution at lower ionic strength and higher temperature, such as 0.2×SSC, 0.1% SDS, 60° C.; or (2) adding denaturant during hybridization, such as 50% (v/v) methylphthalamide, 0.1% calf serum/0.1% Ficoll, 42° C., etc.; or (3) Hybridization occurs only when the identity between the two sequences is at least 90% or more, and more preferably 95% or more.

It should be understood that although the BXL gene of the present invention is preferably derived from Arabidopsis thaliana, other genes from other plants that are highly homologous (such as 80% or more, such as 85%, 90%, 95% or even 98%, 99%, or 100% sequence identity) to the Arabidopsis BXL gene are also within the scope of the present invention. Methods and tools for comparing sequence identity are also well known in the art, such as BLAST.

The full-length BXL nucleotide sequence of the present invention or its fragments can usually be obtained by PCR amplification method, recombination method or artificial synthesis method. For the PCR amplification method, primers can be designed according to the relevant nucleotide sequence disclosed in the present invention, especially the open reading frame sequence, and a commercially available DNA library or a cDNA library prepared according to a conventional method known to those skilled in the art is used as a template to amplify the relevant sequence. When the sequence is long, it is often necessary to perform two or more PCR amplifications, and then splice the amplified fragments together in the correct order. Once the relevant sequence is obtained, the recombination method can be used to obtain the relevant sequence in large quantities. It is usually cloned into a vector, and then transferred into a cell, and then the relevant sequence is isolated from the proliferated host cell by conventional methods.

In addition, artificial synthesis methods can also be used to synthesize related sequences, especially when the fragment length is short. Usually, by first synthesizing multiple small fragments, and then ligating to obtain fragments with very long sequences. At present, the DNA sequence encoding the protein (or fragment or derivative thereof) of the present invention can be obtained completely through chemical synthesis. The DNA sequence can then be introduced into various existing DNA molecules (or such as vectors) and cells known in the art. In addition, mutations can also be introduced into the protein sequence of the present invention through chemical synthesis.

Polypeptide Encoded by BXL Gene

As used herein, the terms “polypeptide of the present invention” and “protein encoded by the BXL gene” can be used interchangeably, and both refer to polypeptides and variants of BXL derived from plants (such as Arabidopsis). In a preferred embodiment, a typical amino acid sequence of the polypeptide of the present invention is shown in SEQ ID NO.:1 (Arabidopsis thaliana).

The present invention relates to a BXL polypeptide and variants thereof for regulating traits. In a preferred embodiment of the present invention, the amino acid sequence of the polypeptide is shown in SEQ ID NO.: 1. The polypeptide of the present invention can effectively regulate the traits of plants (such as monocots or dicots).

The present invention also includes polypeptides or proteins with the same or similar functions that have 50% or more (preferably 60% or more, 70% or more, 80% or more, more preferably 90% or more, more preferably 95% or more, most preferably 98% or more, such as 99% or 100%) homology with the sequence as shown in SEQ ID NO. 1 of the present invention.

The “same or similar function” mainly refers to: “regulate the agronomic traits of plants (such as monocots or dicots)”.

The polypeptide of the present invention can be a recombinant polypeptide, a natural polypeptide, or a synthetic polypeptide. The polypeptide of the present invention may be a natural purified product, or a chemically synthesized product, or produced from a prokaryotic or eukaryotic host (for example, bacteria, yeast, higher plant, insect, and mammalian cells) using recombinant technology. Depending on the host used in the recombinant production protocol, the polypeptide of the present invention may be glycosylated or non-glycosylated. The polypeptide of the present invention may also include or not include the initial methionine residue.

The present invention also includes a BXL protein fragment and analog having BXL protein activity. As used herein, the terms “fragment” and “analog” refer to polypeptides that substantially retain the same biological function or activity as the natural BXL protein of the present invention.

The polypeptide fragments, derivatives or analogs of the present invention may be: (i) polypeptides with one or more conservative or non-conservative amino acid residues (preferably conservative amino acid residues) substituted, and such substituted amino acid residues may or may not be encoded by the genetic code; or (ii) a polypeptide with a substitution group in one or more amino acid residues; or (iii) a polypeptide formed by fusion of a mature polypeptide with another compound (such as a compound that prolongs the half-life of the polypeptide, such as polyethylene glycol); or (iv) a polypeptide formed by fusing an additional amino acid sequence to the polypeptide sequence (such as a leader sequence or secretory sequence or a sequence or proprotein sequence used to purify the polypeptide, or fusion protein). According to the definition herein, these fragments, derivatives and analogs belong to the scope well known to those skilled in the art.

In the present invention, the polypeptide variant is the amino acid sequence as shown in SEQ ID NO.: 1, derived sequence obtained by several (usually 1-60, preferably 1-30, more preferably 1-20, most preferably 1-10) substitutions, deletions or additions of at least one amino acid, and addition of one or several (usually 20 or less, preferably 10 or less, more preferably 5 or less) amino acids at the C-terminus and/or N-terminus. For example, in the protein, when amino acids with similar or close properties are substituted, the function of the protein is usually not changed, and the addition of one or several amino acids to the C-terminal and/or terminal usually does not change the function of the protein. These conservative variants are best produced by substitutions according to Table 1.

TABLE 1

| Initial | Representative | Preferred |
|---|---|---|
| residues | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also includes analogs of the claimed protein. The difference between these analogs and the natural SEQ ID NO.: 1 can be the difference in the amino acid sequence, the difference in the modified form that does not affect the sequence, or both. Analogs of these proteins include natural or induced genetic variants. Induced variants can be obtained by various techniques, such as random mutagenesis by radiation or exposure to mutagens, site-directed mutagenesis or other known molecular biology techniques. Analogs also include analogs having residues different from natural L-amino acids (such as D-amino acids), and analogs having non-naturally occurring or synthetic amino acids (such as β, γ-amino acids). It should be understood that the protein of the present invention is not limited to the representative proteins exemplified above.

Modified (usually without changing the primary structure) forms include: in vivo or in vitro chemically derived forms of proteins such as acetylation or carboxylation. Modifications also include glycosylation, such as those that undergo glycosylation modifications during protein synthesis and processing. This modification can be accomplished by exposing the protein to an enzyme that performs glycosylation (such as a mammalian glycosylase or deglycosylase). Modified forms also include sequences with phosphorylated amino acid residues (such as phosphotyrosine, phosphoserine, and phosphothreonine).

Expression Vector

The present invention also relates to a vector containing the polynucleotide of the present invention, a host cell produced by genetic engineering using the vector of the present invention or the mutant protein coding sequence of the present invention, and a method for producing the polypeptide of the present invention through recombinant technology.

Through conventional recombinant DNA technology, the polynucleotide sequence of the present invention can be used to express or produce recombinant mutant protein. Generally speaking, there are the following steps:

(1) Using the polynucleotide (or variant) of the present invention encoding the protein of the present invention, or using a recombinant expression vector containing the polynucleotide to transform or transduce a suitable host cell;

(2). Host cells cultured in a suitable medium;

(3). Separating and purifying protein from culture medium or cells.

The present invention also provides a recombinant vector including the gene of the present invention. As a preferred way, the downstream of the promoter of the recombinant vector contains a multiple cloning site or at least one restriction site. When it is necessary to express the target gene of the present invention, the target gene is ligated into a suitable multiple cloning site or restriction site, so that the target gene and the promoter are operably linked. As another preferred way, the recombinant vector includes (from 5′ to 3′ direction): a promoter, a target gene, and a terminator. If necessary, the recombinant vector may also include elements selected from the group consisting of: 3′ polynucleotide signal; untranslated nucleic acid sequence; transport and targeting nucleic acid sequence; resistance selection marker (dihydrofolate reductase, neomycin resistant, hygromycin resistance and green fluorescent protein, etc.); enhancer; or operator.

In the present invention, the polynucleotide sequence encoding the protein of the present invention can be inserted into a recombinant expression vector. The term “recombinant expression vector” refers to bacterial plasmids, bacteriophages, yeast plasmids, plant cell viruses, mammalian cell viruses such as adenovirus, retrovirus or other vectors well known in the art. Any plasmid and vector can be used as long as it can be replicated and stabilized in the host. An important feature of an expression vector is that it usually contains an origin of replication, a promoter, a marker gene, and translation control elements.

Methods well known to those skilled in the art can be used to construct an expression vector containing the DNA sequence encoding the protein of the present invention and appropriate transcription/translation control signals. These methods include in vitro recombinant DNA technology, DNA synthesis technology, and in vivo recombination technology. The DNA sequence can be effectively linked to an appropriate promoter in the expression vector to guide mRNA synthesis. Representative examples of these promoters are: Escherichia coli lac or trp promoter; lambda phage PL promoter; eukaryotic promoters include CMV immediate early promoter, HSV thymidine kinase promoter, early and late SV40 promoter, LTRs of retroviruses and some other known promoters that can control gene expression in prokaryotic or eukaryotic cells or viruses. The expression vector also includes a ribosome binding site for translation initiation and a transcription terminator.

Those of ordinary skill in the art can use well-known methods to construct expression vectors containing the genes of the present invention. These methods include in vitro recombinant DNA technology, DNA synthesis technology, and in vivo recombination technology. When constructing a recombinant expression vector using the gene of the present invention, any enhanced, constitutive, tissue-specific or inducible promoter can be added before the transcription initiation nucleotide.

The vector including the gene of the present invention and the expression cassette can be used to transform an appropriate host cell so that the host expresses the protein. The host cell can be a prokaryotic cell, such as Escherichia coli, Streptomyces, Agrobacterium; or a lower eukaryotic cell, such as a yeast cell; or a higher eukaryotic cell, such as a plant cell. Those of ordinary skill in the art know how to select appropriate vectors and host cells. Transformation of host cells with recombinant DNA can be carried out by conventional techniques well known to those skilled in the art. When the host is a prokaryote (such as Escherichia coli), it can be treated with CaCl₂ method or electroporation method. When the host is a eukaryote, the following DNA transfection methods can be selected: calcium phosphate co-precipitation method, conventional mechanical methods (such as microinjection, electroporation, liposome packaging, etc.). Agrobacterium transformation or gene gun transformation can also be used to transform plants, such as leaf disc method, immature embryo transformation method, flower bud soaking method, etc. The transformed plant cells, tissues or organs can be regenerated by conventional methods to obtain transgenic plants.

In addition, the expression vector preferably contains one or more selectable marker genes to provide phenotypic traits for selection of transformed host cells, such as dihydrofolate reductase for eukaryotic cell culture, neomycin resistance, and green fluorescent protein (GFP), or tetracycline or ampicillin resistance for E. coli.

A vector containing the above-mentioned appropriate DNA sequence and an appropriate promoter or control sequence can be used to transform an appropriate host cell so that it can express the protein.

The host cell can be a prokaryotic cell, such as a bacterial cell; or a lower eukaryotic cell, such as a yeast cell; or a higher eukaryotic cell, such as a mammalian cell. Representative examples are: Escherichia coli, Streptomyces; bacterial cells of Salmonella typhimurium; fungal cells such as yeast and plant cells (such as rice cells).

When the polynucleotide of the present invention is expressed in higher eukaryotic cells, if an enhancer sequence is inserted into the vector, the transcription will be enhanced. Enhancers are cis-acting factors of DNA, usually about 10 to 300 base pairs, acting on promoters to enhance gene transcription. Examples include the 100 to 270 base pair SV40 enhancer on the late side of the replication initiation point, the polyoma enhancer on the late side of the replication initiation point, and adenovirus enhancers.

Those of ordinary skill in the art know how to select appropriate vectors, promoters, enhancers and host cells.

Transformation of host cells with recombinant DNA can be carried out by conventional techniques well known to those skilled in the art. When the host is a prokaryotic organism such as Escherichia coli, competent cells that can absorb DNA can be harvested after the exponential growth phase and treated with the CaCl₂ method. The steps used are well known in the art. Another method is to use MgCl₂. If necessary, the transformation can also be carried out by electroporation. When the host is a eukaryote, the following DNA transfection methods can be selected: calcium phosphate co-precipitation method, conventional mechanical methods such as microinjection, electroporation, liposome packaging, etc.

The obtained transformants can be cultured by conventional methods to express the polypeptide encoded by the gene of the present invention. Depending on the host cell used, the medium used in the culture can be selected from various conventional mediums. The culture is carried out under conditions suitable for the growth of the host cell. When the host cell has grown to an appropriate cell density, a suitable method (such as temperature conversion or chemical induction) is used to induce the selected promoter, and the cell is cultured for a period of time.

The recombinant polypeptide in the above method can be expressed in the cell or on the cell membrane, or secreted out of the cell. If necessary, the physical, chemical, and other characteristics can be used to separate and purify the recombinant protein through various separation methods. These methods are well known to those skilled in the art. Examples of these methods include but are not limited to: conventional renaturation treatment, treatment with protein precipitation agent (salting out method), centrifugation, bacteria broken through osmotic, ultra-treatment, ultra-centrifugation, molecular sieve chromatography (gel filtration), adsorption chromatography, ion exchange chromatography, high performance liquid chromatography (HPLC) and various other liquid chromatography techniques and combinations of these methods.

Agricultural Preparations

The active substance of the present invention (such as the inhibitor of BXL gene or an encoded protein thereof) can be prepared into agricultural preparations by conventional methods, such as solutions, emulsions, suspensions, powders, foams, pastes, granules, aerosol, natural and synthetic materials impregnated with active substances, microcapsules in polymers, coating agent for seeds.

These preparations can be produced by known methods, for example, mixing active substances with extenders, which are liquid or liquefied gases or solid diluents or carriers, and can choose the surface active agent arbitrarily, namely emulsifier and/or dispersant and/or foam forming agent. For example, when water is used as an extender, organic solvents can also be used as additives.

When a liquid solvent is used as a diluent or carrier, it is basically suitable, such as: aromatic hydrocarbons, such as xylene, toluene or alkyl naphthalene; chlorinated aromatic or chlorinated aliphatic hydrocarbons, such as chlorobenzene, chloroethylene or dichloromethane; aliphatic hydrocarbons, such as cyclohexane or paraffin, such as mineral oil fractions; alcohols, such as ethanol or ethylene glycol and their ethers and lipids; ketones, such as acetone, methyl ethyl ketone, methyl isobutyl ketone or cyclohexanone; or uncommon polar solvents such as dimethylformamide and dimethyl sulfoxide, and water.

As far as the diluent or carrier of liquefied gas is concerned, it refers to a liquid that will become a gas at normal temperature and pressure, such as aerosol propellants, such as halogenated hydrocarbons, butane, propane, nitrogen, and carbon dioxide.

The solid carrier can be ground natural minerals, such as kaolin, clay, talc, quartz, activated clay, montmorillonite, or diatomaceous earth, and ground synthetic minerals, such as highly dispersed silicic acid, aluminium oxide and silicate. The solid carrier for particles is crushed and classified natural zircon, such as calcite, marble, pumice, sepiolite and dolomite, as well as particles synthesized from inorganic and organic coarse powder, and organic materials such as sawdust, coconut shell, maize cob and tobacco stem particles.

Nonionic and anionic emulsifiers can be used as emulsifiers and/or foam forming agent. For example, polyoxyethylene-fatty acid esters, polyoxyethylene-fatty alcohol ethers, such as alkyl aryl polyglycol ethers, alkyl sulfonates, alkyl sulfates, aryl sulfonates and albumin hydrolysate. Dispersants include, for example, lignin sulfite waste liquor and methyl cellulose.

Binders such as carboxymethyl cellulose and natural and synthetic polymers in powder, granule or emulsion form, such as arabic gum, polyvinyl alcohol and polyvinyl acetate can be used in the formulation.

Colorants such as inorganic dyes such as iron oxide, diamond oxide and Prussian blue; organic dyes such as organic dyes such as azo dyes or metal phthalocyanine dyes; and trace nutrients such as iron, manganese, boron, and copper, cobalt, aluminum and zinc salts.

In the present invention, the “agricultural preparation” is usually an agricultural plant growth regulator, which contains the inhibitor of the BXL gene or an encoded protein thereof as an active ingredient for improving plant traits (e.g., enhancement of plant stress resistance (such as drought resistance, salt tolerance, osmotic pressure resistance, heat resistance, etc.); and/or resistance to pathogenic bacteria; and/or reducing lignin content, increasing fiber and pectin content; and/or delaying seed germination; and/or delaying flowering time; and/or reducing stomatal aperture; and/or improving the palatability of feed; and an agriculturally acceptable carrier.

As used herein, the “agriculturally acceptable carrier” is an agrochemically acceptable solvent, suspending agent or excipient used to deliver the active substance of the present invention to plants. The carrier can be liquid or solid. The agriculturally acceptable carrier suitable for the present invention is selected from the group consisting of water, buffer, DMSO, surfactants such as Tween-20, and a combination thereof. Any agriculturally acceptable carrier known to those skilled in the art can be used in the present invention.

The agricultural preparation of the present invention may include a feed composition, an organic fertilizer composition, or a pesticide composition.

In a preferred embodiment, the agricultural preparation of the present invention includes a solid feed composition or a liquid feed composition.

In a preferred embodiment, the feed composition of the present invention is a plant breeding additive.

The agricultural preparation of the present invention can be combined with other substances that improve the palatability of feed (such as cellulose hydrolase enzyme, pectin synthase, pectin additive, galactase, ethylene molecule, abscisic acid, etc.).

The agricultural preparations of the present invention can be prepared as a mixture with other anti-stress agents (such as drought-resistant agents, salt-tolerant agents, osmotic pressure-resistant agents, heat-resistant agents, etc.) and present in their commercial preparations or in dosage forms prepared from these preparations, these other drought-resistant agents include (but are not limited to): drought-resistant seed coating agents, drought-resistant water-retaining agents, or drought-resistant sprays, etc.; these other salt-tolerant agents include (but are not limited to): salt-tolerant microbial agents, salt-tolerant thickener; these other osmotic pressure agents include (but are not limited to): sprays, biological protein protectors, trehalose; these other heat-resistant agents include (but are not limited to): heat-resistant film, biological wax preparations, hydration film, trehalose.

The agricultural preparations of the present invention are prepared as a mixture with other anti-pathogenic agents and are present in their commercial preparations or in dosage forms prepared from these preparations. These other anti-pathogenic agents include (are not limited to): saponins, Phenolics, disease-fighting compounds, organic sulfides, unsaturated fatty acids.

In addition, the agricultural preparations of the present invention can also be prepared as a mixture with synergists and present in their commercial preparations or in dosage forms prepared from these preparations. These synergists are compounds that enhance the effect of the active compound. It is active by itself, and there is no need to add a synergist.

The dosage form of the agricultural preparation of the present invention can be various, as long as the active ingredient can effectively reach the dosage form of the plant body, it is all right. From the standpoint of easy preparation and application, the preferred agricultural preparation is a spray or solution formulation.

The agricultural formulation of the present invention usually contains 0.0001-99 wt %, preferably 0.1-90 wt % of the active ingredient of the present invention, which accounts for the total weight of the agricultural formulation. The concentration of the active ingredient of the present invention in the commercial preparation or use dosage form can vary within a wide range. The concentration of the active ingredient of the present invention in the commercial preparation or use dosage form can be from 0.0000001-100% (g/v), preferably between 0.0001 and 50% (g/v).

Improvement of Plant (Such as Monocots or Dicots) Traits

The present invention also provides a method for improving the traits of plants (such as monocots or dicots). The improvement includes: (i) enhancing plant resistance; and/or (ii) resistance to pathogenic bacteria; and/or (iii) reducing the content of lignin and increasing the content of fiber and pectin, including the steps of reducing the expression level and/or activity of the BXL gene or an encoded protein thereof in the plant, or adding an inhibitor of the BXL gene or an encoded protein thereof.

In the present invention, other substances that can (i) enhance plant resistance to stress; and/or (ii) resist pathogenic bacteria; and/or (iii) reduce lignin content and increase fiber and pectin content can be further used in conventional methods to treat plants or plants seeds to improve the traits of corresponding plants.

The main advantages of the present invention include:

(1) The present invention screens for the first time a BXL gene, which encodes a glycosyl hydrolase, which can improve plant traits by inhibiting the expression or activity of the BXL gene or an encoded protein thereof.

(2) The present invention has found for the first time that reducing the expression of the BXL gene or an encoded protein thereof can significantly (i) enhance plant stress resistance; and/or (ii) resist pathogenic bacteria; and/or (iii) reduce lignin content and increase fiber and pectin content.

(3) The present invention has found for the first time that reducing the expression of the BXL gene or an encoded protein thereof can improve the palatability of feed.

(4) The present invention has found for the first time that reducing the expression of the BXL gene or an encoded protein thereof can also (a) delay seed germination; (b) delay flowering time; (c) reduce stomatal aperture; (d) enhance plant resistance to stress; (e) enhance the resistance of plants to disease bacteria; (f) improve the palatability of plants as feed.

The present invention will be further explained below in conjunction with specific embodiments. It should be understood that these embodiments are only used to illustrate the present invention and not to limit the scope of the present invention. The experimental methods without specific conditions in the following examples are usually in accordance with conventional conditions such as Sambrook et al., Molecular Cloning: Laboratory Manual (New York: Cold Spring Harbor Laboratory Press, 1989), or in accordance with the conditions described in the manufacturer The suggested conditions. Unless otherwise specified, the materials and reagents used in the examples are all commercially available products.

### Example 1

The Arabidopsis thaliana models involved in the following examples are Col-0, SALK_012090C (bxl1-1), and SALK_086578C (bxl1-2) from the Arabidopsis Biological Resource Center (ABRC).

Taking Arabidopsis as an example.

1. Obtaining of Mutants

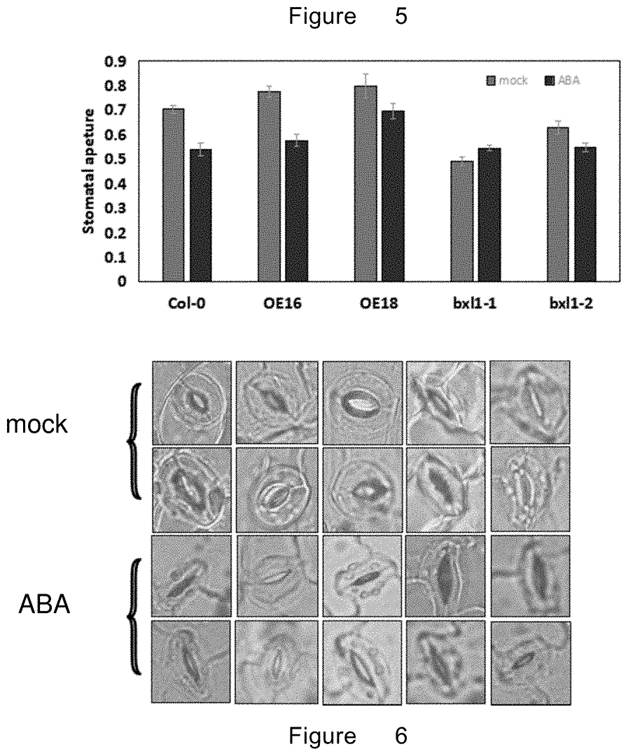

From the data of genome-wide transcript level sequencing induced by Bacillus amyloliquefaciens strain GB03 (a commercial model of beneficial plant rhizosphere growth-promoting bacteria that promotes plant growth, purchased from ATCC, Global Biological Resource Center), a significantly down-regulated gene, beta-xylosidase 1 (BXL1) has been found. The seed number of the T-DNA insertion mutant of this gene was found on the Tair website, and two T-DNA insertion mutants, SALK_0120909C and SALK_86578C, were ordered from the Arabidopsis Biological Resource Center (ABRC). After genotype identification and confirmation, it is observed that the BXL1 gene loss-of-function mutant exhibits delayed germination, slightly slower vegetative growth and delayed flowering time during the growth process. The results are shown in FIG. 4, (A) shows BXL1 mutant plants can have a slow seed germination rate. Figure (B) shows that the mutant plants show a slow growth rate. In the same growth period, compared with the wild type, the BXL1 mutant shows a smaller plant morphology, and the flowering time is slower. At the beginning of fruiting, the length of the fruit pod is shorter than that of the wild type.

Compared with the wild type, the BXL1 loss-of-function plants show a smaller leaf area at the same growth period, but when the mutant reaches the flowering stage, the leaf area of the plant does not decrease compared with the wild type at the same period; after bolting and blooming, the mutant shows shorter stems and smaller fruit pods. However, after maturity, the biomass of the plant and the total seed weight of a single plant do not show significant differences compared with the wild type, as shown in FIG. 5. FIG. 5, (A) (B), the results of the two graphs show that the BXL1 gene has no effect on the final biomass per plant of a plant and the thousand-seed weight of a single plant seed. Figure (A) shows that when the BXL1 mutant plant reaches the flowering stage, there is no significant change in the dry weight of a single plant compared to the wild type when it reaches the flowering stage. Figure (B) show the final harvested seeds of the mutant plants do not show a difference in the thousand-seed weight of a single plant seed compared to the wild-type single plant harvested seeds.

2. Transgene to Obtain Over-Expression Materials

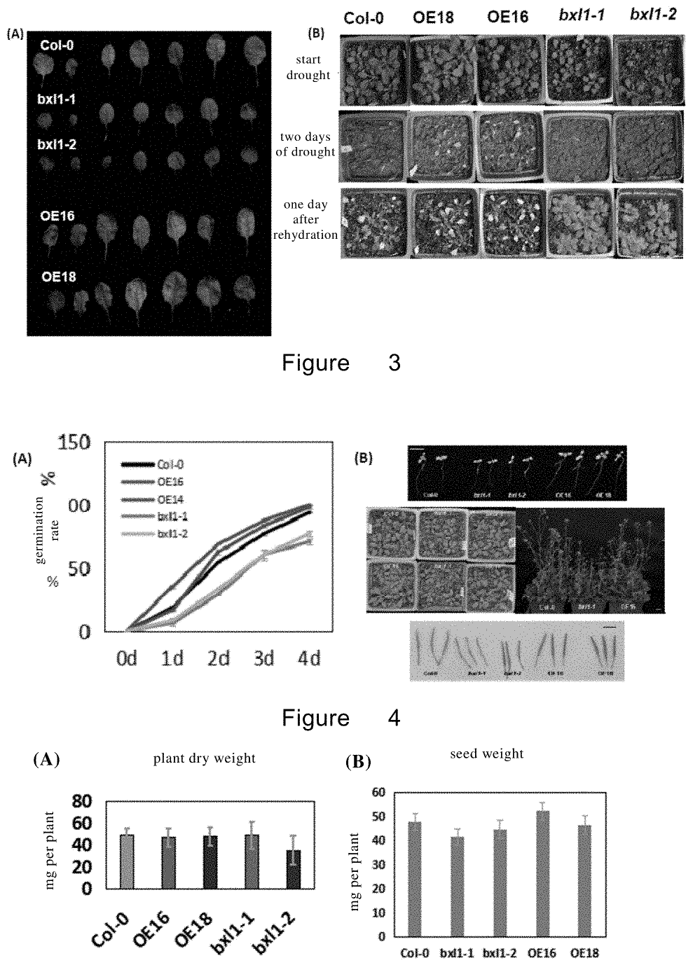

Using transgenic technology, Pro_{BXL1}:GUS and 35S:BXL1:flag materials were constructed. Taking the 2.8k upstream of the start codon of BXL1 as its promoter analysis (not including the 5′ UTR region), and fuse it into pGWB533 by the Getway method. The full-length BXL1 CDs were fused into pGWB511 by Getway method. Transcription level detection of the expression of BXL1 in different materials, the result is shown in FIG. 2, quantitative PCR detects the expression level of the BXL1 gene at the transcript level of the BXL1 mutant or overexpression lines. The results show that for overexpressed plants, the expression of BXL1 gene is significantly increased, compared with wild-type plants. However, the expression of the mutant BXL1 gene is significantly suppressed.

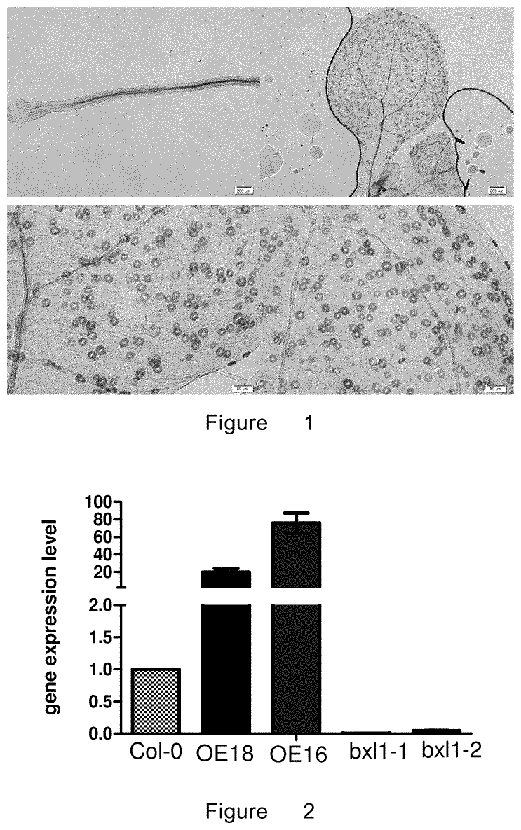

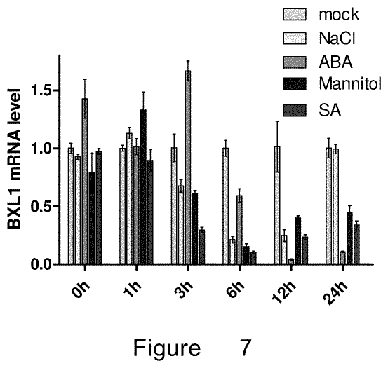

The primers used for detection are: F 5′-AGTTGATGTTGATGCTTGCA-3′ (SEQ ID NO.: 3), R 5′-AAGTTGCGGTTGGACCAAAA-3′ (SEQ ID NO.: 4). GUS staining by histochemical method shows that BXL1 can be specifically expressed in the stomata. As shown in FIG. 1, the BXL1 gene can be specifically expressed in the stomata. Under microscope observation, the number of stomata of the mutant and overexpression materials does not change compared with the wild-type material, but the stomatal aperture is smaller in the mutant material. The data is shown in FIG. 6. FIG. 6 shows that the BXL1 gene regulates the opening of plant stomata. Compared with the wild type, the stomatal aperture in the BXL1 mutant is significantly smaller. Stomatal opening experiments were performed to detect different genotypes. Calculating the opening of fifty stomata (the opening width of the stomata) for each genotype as a technical repetition. The independent biological experiment was repeated three times, and the results are consistent.

1. Stress Treatment of Drought and Pathogenic Bacteria

In view of the tissue expression specificity and growth phenotype of BXL1, the effects of biotic and abiotic stress factors (salicylic acid SA, salt NaCl, mannitol Mannitol, abscisic acid ABA) on the transcription level of BXL1 gene were tested and has found that these factors can significantly down-regulate BXL1. The results are shown in FIG. 7. The BXL1 gene is regulated by a variety of stress factors. Salicylic acid, salt, abscisic acid, mannitol, these stress factors can significantly down-regulate the expression of BXL1 gene in a specific time. The mock in the figure is used as a control group, which means water treatment, because all the compound solvents used in the experiment are water. This result shows that BXL1 can also resist high salt stress and osmotic stress in addition to being resistant to drought and pathogenic bacteria. The specific operation is: there is also 1% sucrose (weight-volume ratio) 1/2MS medium to cultivate 14-day-old wild Arabidopsis seedlings, 2 μM SA, 300 mM NaCl, 400 mM Mannitol, 100 μM ABA aqueous solution evenly sprayed on the surface of the leaves, water (containing the same volume of absolute ethanol added to other treatments) as a control, putting it back to the original growth environment, collecting samples within a specific time, and performing downstream gene expression testing. Based on this, experiments on drought and pathogenic bacteria were carried out. It is found that the loss-of-function mutant bxl1 can better resist drought, and at the same time can enhance the resistance to Pseudomonas syringae Pst DC3000 (a commercial model pathogen, purchased from ATCC, Global Biological Resource Center). The results are shown in FIG. 3. FIG. 3, (A) and (B), the results of the two graphs show the basic phenotype of BXL1 gene resistance to pathogenic bacteria and drought. Figure (A) shows that BXL1 mutant plants can have better resistance to pathogenic bacteria, and the degree of infection is slower than that of the wild type. The specific method of drought treatment is: the nutrient soil and vermiculite are mixed evenly according to the volume ratio of 1:2, and the same volume of mixed soil is evenly filled into each bowl, and then watered in the tray, so that each bowl with the same soil will evenly absorb the water. Picking out 7-day-old seedlings of the same size and planting them in the prepared soil, putting them under long-day conditions (16 hours of light, 8 hours of darkness, and light intensity of 220 μm·s⁻¹), and watering them normally for 10 days, then stopping watering and continuously recording the drought phenotype. After the drought phenotype appears, it can be rehydrated, recording phenotype. The results are shown in FIG. 3-(B). Figure (B) shows that the mutant plants can better resist the drought environment. Under the same drought conditions, the mutant can maintain a higher survival rate. The details of the disease resistance experiment are: wild-type, mutant and over-expression transgenic plants are planted under short-day conditions (14 hours of light, 10 hours of darkness, 110 μm·s⁻¹) to 4 weeks in size, and Pst DC3000 is activated in a 28 degree incubator in advance, using sterile 10 mM MgCl₂ to suspend DC3000 to a final concentration of 5×10⁷ cfu/mL (about OD=0.1), and adding the surfactant Silwet L-77 with a final concentration of 0.02% (V/V). The bacterial solution is evenly sprayed on the plant leaves, and then attached with plastic wrap to keep for 6 hours. After 3 days, random samples are taken for quantitative analysis. After 5 days, the infection phenotype of pathogens is recorded. The results are shown in FIG. 3-(B). The drought experiment is independently repeated 4 times, and the results show a consistent phenotype. FIG. 3-(A), the disease resistance experiment is repeated twice independently, and the results show the same.

### Example 2

BXL1 has homologous genes in many dicotyledonous plants, such as alfalfa, apple, flax, rape, radish, pepper, cherry, Chinese date, Brasenia schreberi and alfalfa. In alfalfa, the gene highly homologous to BXL1 is named Medtr2g034720.1. The invention realizes the expression regulation of the Medtr2g034720.1 gene in alfalfa through gene editing technology, so as to change the cell wall composition of alfalfa, regulate the palatability of alfalfa as a feed, and at the same time enhance the ability of alfalfa to resist biotic and abiotic stress.

CRISPR (Clustered Regularly Interspaced Short Palindromic Repeats) is a very powerful gene editing technology that has been widely used in animals and plants. The experimental design of the present invention selects the psgR-Cas9-At carrier, and designs the sg-RNA online at www.atum.bio/eCommerce/cas9/input. In order to effectively construct the target fragment of the present invention on the psgR-Cas9-At vector, we synthesized two types of oligonucleotide linkers, namely: 5′-GATTGNNNNNNNNNNNNNNNNNNN-3′(SEQ ID NO.: 5) and 5′-TGGCGNNNNNNNNNNNNNNNNNNN-3′(SEQ ID NO.: 6). The reverse is 3′-CNNNNNNNNNNNNNNNNNNNCAAA-5 (SEQ ID NO.: 7). The process of oligonucleotide annealing and cloning into the backbone vector is as follows:

First, using BbsI f to digest the psgR-Cas9-At/Os vector at 37° C. for 30 minutes:

1 μg psgR-Cas9-At/Os
1 ul FastDigest BbsI (Fermentas)
1 ul FastAP (Fermentas)
2 ul 10X FastDigest Buffer
X ul ddH2O
Total 20 ul

Then the digested product is purified and recovered. Then each pair of oligonucleotides is phosphorylated and annealed. The reaction system is:

1 ul
oligo 1 (100 μM)
1 ul oligo 2 (100 μM)
1 ul 10X T4 Ligation Buffer * (NEB)
6.5 ul ddH2O
0.5 ul T4 PNK (NEB)
10 ul total

The thermal cycle is 37 degrees for 30 minutes, 95 degrees for five minutes and then decreases to 25 degrees at 5 degrees per minute. Finally, the linearized vector backbone and the processed oligonucleotides are connected with T4 ligase. Connected for ten minutes at room temperature, the reaction system is as follows:

X μL of psgR-Cas9-At/Os (50 ng) digested with BbsI from step 2 (X ul BbsI digested psgR-Cas9-At/Os from step 2 (50 ng))

1 ul phosphorylated and annealed oligo duplex from step 3 (1:200
dilution)
5 ul 2X Quickligation Buffer (NEB)
X ul ddH_{₂}O
Subtotal 10 ul
1 ul Quick Ligase (NEB)
Total 11 ul

Finally, the ligated product was transformed into E. coli. A medium containing 50 micrograms per milliliter of ampicillin antibiotics was used to screen positive single clones. Extracting plasmids from positive clones and sending them for sequencing. Making sure that the target gene has been connected to the vector.

Alfalfa plants with Medtr2g034720.1 gene knocked out by CRISPR technology are expected to produce changes in growth phenotype, such as delayed germination and prolonged flowering time and the like. However, the overall biomass will not decrease. As the main material of feed, alfalfa plants lacking the Medtr2g034720.1 gene will have a different cell wall composition than wild-type plants. The main manifestation is that the content of lignin may be less, and the content of fiber and pectin will increase, thereby improving its palatability as feed. At the same time, alfalfa lacking the Medtr2g034720.1 gene can produce better resistance to drought and pathogenic bacteria, thereby improving the growth ability of alfalfa and expanding the growth range of alfalfa. This provides strong support for the development of alfalfa-based feed industry in my country.

All publications mentioned herein are incorporated by reference as if each individual document was cited as a reference, as in the present application. It should also be understood that, after reading the above teachings of the present invention, those skilled in the art can make various changes or modifications, equivalents of which falls in the scope of claims as defined in the appended claims.

1. (canceled)

2. A method for the improvement of plant traits, said traits selected from the group consisting of:
(i) enhancing plant stress resistance;
(ii) enhancing plant resistance to pathogenic bacteria
(iii) reducing the lignin content of the plant, and
(iv) increasing the fiber or pectin content of the plant.
the method comprising preparing a composition comprising an inhibitor of a BXL gene or an encoded protein thereof, and agronomically acceptable carrier, and administering said composition to a plant.

3. The method of claim 2, wherein the composition or preparation is also used for one or more purposes selected from the group consisting of:
(a) delaying seed germination;
(b) delaying flowering time;
(c) reducing the stomatal aperture;
(d) enhancing resilience to drought, salt or osmotic stress;
(e) enhancing the ability to resist pathogenic bacteria;
(f) improving the palatability as feed.

4. The method of claim 2, wherein the plant is selected from a herbaceous plant and a woody plant.

5. The method of claim 4, wherein the herbaceous plant is selected from the group consisting of Solanaceae, Gramineous plant, Leguminous plant, Cruciferous plant, and a combination thereof.

6. The method of claim 4, wherein the woody plant is selected from the group consisting of Actinidiaceae, Rosaceae, Moraceae, Capparaceae, Rutaceae, Malvaceae, Rosaceae, Hibiscus, and a combination thereof.

7. The method of claim 2, wherein the plant is selected from the group consisting of Arabidopsis, alfalfa, apple, Camelina sativa, soybean, rice, oilseed rape, radish, pepper, cherry, Jujube, cabbage, Cleome hassleriana, citrus, durian, and a combination thereof.

8. The method of claim 2 use of claim 1, wherein the BXL gene is selected from the group consisting of: BXL1, BXL2, BXL3, BXL4, BXL7, AT5G10560, AT3G19620, AT5G09700, and a combination thereof.

9. The method of claim 2, wherein the BXL gene is derived from one or more plants selected from the group consisting of Arabidopsis, alfalfa, apple, Camelina sativa, soybean, oilseed rape, radish, pepper, cherry, Jujube, cabbage, Cleome hassleriana, citrus, durian.

10. The method of claim 2, wherein the amino acid sequence of the encoded protein of the BXL gene is selected from the group consisting of:
(i) a polypeptide having the amino acid sequence as shown in SEQ ID NO.:1;
(ii) a polypeptide derived from (i), having the function of regulating agronomic traits formed by the substitution, deletion or addition of 1-10 amino acid residues of the amino acid sequence as shown in SEQ ID NO.:1; or (iii) a polypeptide whose amino acid sequence is greater than or equal to 90% homologous to the amino acid sequence as shown in SEQ ID NO.:1 and having the function of improving plant agronomic traits.

11. The method of claim 2, wherein the nucleotide sequence of the BXL gene is selected from the group consisting of:
(a) a polynucleotide encoding the polypeptide as shown in SEQ ID NO.:1;
(b) a polynucleotide whose sequence is shown in SEQ ID NO.: 2;
(c) a polynucleotide whose nucleotide sequence has a homology of ≥95% to SEQ ID NO.: 2;
(d) a polynucleotide of 1-60 nucleotides which is truncated or elongated at the 5′ end and/or 3′ end of the polynucleotide as shown in SEQ ID NO.: 2; and
(e) a polynucleotide complementary to any of the polynucleotides as described in (a) to (d).

12. A composition comprising:
(a) an inhibitor of a BXL gene or an encoded protein thereof; and
(b) an agronomically acceptable carrier.

13. The composition of claim 12, wherein the composition contains 0.0001-99 wt %, preferably 0.1-90 wt % of component (a), based on the total weight of the composition.

14. The composition of claim 12, wherein the composition further includes other substances that improve the palatability of feed.

15. The composition of claim 12, wherein the composition further includes other substances that enhance plant resistance to stress.

16. The composition of claim 12, wherein the composition also includes other substances that reduce the content of lignin and increase the content of fiber and pectin.

17. (canceled)

18. A method for improving the palatability of feed, comprising the steps: reducing the expression level and/or activity of the BXL gene or the encoded protein thereof in the plant, thereby improving the palatability of feed.

19. The method of claim 18, wherein the reducing the expression or activity of the BXL gene or the encoded protein thereof in the plant is achieved by a method selected from the group consisting of gene mutation, gene knockout, gene disruption, RNA interference, Crispr technology, ZFN, TALEN, and a combination thereof.

20-23. (canceled)

24. A method for preparing a genetically engineered plant tissue or plant cell, comprising the steps:
reducing the expression and/or activity of the BXL gene or the encoded protein thereof in a plant tissue or plant cell, thereby obtaining a genetically engineered plant tissue or plant cell; the method further comprising introducing the inhibitor of the BXL gene or the encoded protein thereof into the plant tissue or plant cell.

25. A method for preparing a genetically engineered plant, comprising the steps: regenerating into a plant, the genetically engineered plant tissue or plant cell prepared by the method of:
reducing the expression and/or activity of the BXL gene or the encoded protein thereof in a plant tissue or plant cell, thereby obtaining a genetically engineered plant tissue or plant cell; the method further comprising introducing the inhibitor of the BXL gene or the encoded protein thereof into the plant tissue or plant cell,
thereby obtaining a genetically engineered plant.
